(19) United States
(12) Patent Application Publication
(10) Pub. No.: US 2024/0310395 A1
(43) Pub. Date: Sep. 19, 2024

(54) DIAGNOSTIC LABORATORY SYSTEMS AND METHODS OF OPERATING

(71) Applicant: SIEMENS HEALTHCARE DIAGNOSTICS INC., Tarrytown, NY (US)

(72) Inventors: Yao-Jen Chang, Princeton, NJ (US); Rayal Raj Prasad Nalam Venkat, Princeton, NJ (US); Benjamin S. Pollack, Jersey City, NJ (US); Vivek Singh, Princeton, NJ (US); Venkatesh NarasimhaMurthy, Hillsborough, NJ (US); Ankur Kapoor, Plainsboro, NJ (US)

(73) Assignee: Siemens Healthcare Diagnostics Inc., Tarrytown, NY (US)

(21) Appl. No.: 18/546,959

(22) PCT Filed: Feb. 17, 2022
(86) PCT No.: PCT/US2022/070700

§ 371 Date: Aug. 17, 2023

Related U.S. Application Data
(60) Provisional application No. 63/150,876, filed on Feb. 18, 2021.

(51) Int. Cl. G01N 35/00; G01N 35/10

(52) U.S. Cl. CPC G01N 35/00712; G01N 35/1016; G01N 2035/1025

(57) ABSTRACT

Methods of operating a diagnostic laboratory system include providing a module configured to perform a function on an item in the diagnostic laboratory system; providing a plurality of sensors, each of the plurality of sensors configured to monitor the function or the item and generate sensor data in response to the monitoring; checking an operational status of a first sensor of the plurality of sensors; receiving sensor data from at least one of the plurality of sensors; and scaling sensor data from the first sensor in response to the operational status and the sensor data to generate revised sensor data. Systems including a middleware server configured to carry out the methods are provided as are other aspects.

### CROSS REFERENCE TO RELATED APPLICATION

This application claims the benefit of U.S. Provisional patent application No. 63/150,876, entitled “DIAGNOSTIC LABORATORY SYSTEMS AND METHODS OF OPERATING” filed Feb. 18, 2021, the disclosure of which is hereby incorporated by reference in its entirety for all purposes.

### FIELD

Embodiments of the present disclosure relate to diagnostic imaging systems of diagnostic laboratory systems and methods of operating such diagnostic imaging systems.

### BACKGROUND

Diagnostic laboratory systems include instruments that conduct clinical chemistry or assays to identify analytes or other constituents in biological specimens (specimens) such as blood serum, blood plasma, urine, interstitial liquid, cerebrospinal liquids, and the like.

Improvements in diagnostic laboratory systems have been accompanied by corresponding advances in automated pre-analytical specimen processing such as centrifugation of specimens to separate specimen constituents, cap removal (decapping) to facilitate specimen access, aliquot preparation, and pre-screening for hemolysis, icterus, and/or lipemia, or normality (HILN), and/or the presence of an artifact in the specimen such as a clot, bubble, or foam. One or more sensors, such as imaging devices and pressure sensors, may monitor the analysis or other processes performed within the diagnostic laboratory system.

### SUMMARY

According to a first aspect, a method of operating a diagnostic laboratory system is provided. The method includes providing a module configured to perform a function on an item in the diagnostic laboratory system; providing a plurality of sensors, each of the plurality of sensors configured to monitor the function or the item and generate sensor data in response to the monitoring; checking an operational status of a first sensor of the plurality of sensors; receiving sensor data from at least one of the plurality of sensors; and scaling sensor data from the first sensor in response to the operational status and the sensor data to generate revised sensor data.

In a further aspect, a diagnostic laboratory system is provided. The diagnostic laboratory system includes a module configured to perform a function on an item in the module; a plurality of sensors, each of the plurality of sensors configured to monitor the function or the item and generate sensor data in response to the monitoring; and a computer configured to: check an operational status of a first sensor; receive sensor data from at least one of the plurality of sensors; and scale sensor data generated by the first sensor in response to the operational status and the sensor data to generate revised sensor data.

In another aspect, a method of operating a diagnostic laboratory system is provided. The method includes providing a module configured to perform an analysis on a specimen in the diagnostic laboratory system; providing a plurality of sensors, each of the plurality of sensors configured to monitor the specimen during the analysis; checking an operational status of a first sensor of the plurality of sensors; receiving sensor data from at least one of the plurality of sensors; and scaling sensor data from the first sensor in response to the operational status and the sensor data to generate revised sensor data.

Still other aspects, features, and advantages of this disclosure may be readily apparent from the following description and illustration of a number of example embodiments, including the best mode contemplated for carrying out the disclosure. This disclosure may also be capable of other and different embodiments, and its several details may be modified in various respects, all without departing from the scope of the disclosure. This disclosure is intended to cover all modifications, equivalents, and alternatives falling within the scope of the claims and their equivalents.

### BRIEF DESCRIPTION OF THE DRAWINGS

The drawings, described below, are provided for illustrative purposes, and are not necessarily drawn to scale. Accordingly, the drawings and descriptions are to be regarded as illustrative in nature, and not as restrictive. The drawings are not intended to limit the scope of the disclosure in any way.

FIG. 1 illustrates a block diagram of a diagnostic laboratory system including a plurality of modules and instruments according to one or more embodiments.

FIG. 2A illustrates a block diagram of a system showing an interaction of a sensor, a sensor check program, a sensor scaling program, and a user configuration program implemented in a diagnostic laboratory system according to one or more embodiments.

FIG. 2B illustrates a block diagram of a system showing an interaction of a plurality of sensors, a sensor check program, a sensor scaling program, and a user configuration program implemented in a diagnostic laboratory system according to one or more embodiments.

FIG. 3A illustrates a top view of a module of a diagnostic laboratory system implemented as an imaging module including three imaging devices according to one or more embodiments.

FIG. 3B illustrates the imaging module of FIG. 3A showing an imaging device in a malfunctioning state according to one or more embodiments.

FIG. 3C illustrates the imaging module of FIG. 3A showing two imaging devices in malfunctioning states according to one or more embodiments.

FIG. 4A illustrates a side elevation view of a capped specimen container containing a specimen, wherein the specimen container is configured to be transported throughout a diagnostic laboratory system according to one or more embodiments.

FIG. 4B illustrates a side elevation view of an uncapped specimen container containing a specimen, wherein the specimen container is configured to be transported throughout a diagnostic laboratory system according to one or more embodiments.

FIG. 5 illustrates a block diagram of an aspiration and dispensing module implemented in a diagnostic laboratory system according to one or more embodiments.

FIG. 6 is a graph illustrating pressure traces of a specimen aspiration by a pipette assembly of an aspiration module aspirating a specimen showing a functioning pressure sensor trace and a malfunctioning pressure sensor trace (dotted) according to one or more embodiments.

FIG. 7 is a flowchart illustrating a method of operating a diagnostic laboratory system according to one or more embodiments.

FIG. 8 is a flowchart illustrating another method of operating a diagnostic laboratory system according to one or more embodiments.

### DETAILED DESCRIPTION

As discussed above, diagnostic laboratory systems, such as automated diagnostic laboratory systems, include instruments that conduct clinical chemistry and/or assays to identify analytes or other constituents in biological specimens. The specimens are typically stored in specimen containers wherein the specimen containers are transported to specific instruments and/or modules within the diagnostic laboratory systems for processing and/or testing.

Some diagnostic laboratory systems perform pre-analytical specimen and/or specimen container processing. For example, some modules in diagnostic laboratory systems may perform centrifugation of specimens to separate specimen constituents. Some modules in diagnostic laboratory systems may perform removal of caps from tube portions of the specimen containers to enable access to specimens located in the specimen containers. Some modules may perform aliquot preparation. Other modules may pre-screen specimens for HILN, and/or the presence of an artifact in the specimen such as a clot, bubble, or foam. Some modules may use one or more sensors, such as imaging devices, coupled to a computer to perform the above-described processes. For example, imaging devices may capture images of the specimens and/or the specimen containers and the computer may analyze the image data generated by the imaging devices to perform the above-described processes.

In some embodiments the diagnostic analyzer systems include modules containing clinical chemistry and/or assay instruments configured to perform analytical tests the specimens. The testing may involve reactions that generate changes, such as fluorescence or luminescence emissions that may be read to determine a presence and/or a concentration of an analyte or other constituent contained in the specimen. Some modules may include one or more sensors, such as one or more imaging devices, coupled to a computer, wherein the computer analyzes image data generated by the one or more imaging devices to determine the concentration and/or presence of analytes.

As stated above, the diagnostic laboratory systems may include a plurality of sensors, which may include the aforementioned imaging devices. Some diagnostic laboratory systems may include one or more pressure sensors configured to measure aspiration and/or dispense pressure, such as pressure in a pipette assembly, during aspiration and/or dispense processes. Temperature sensors may measure temperatures of specimens, analytes, incubation devices, machinery, and other components. Voltage sensors may measure voltages of various machinery and/or specimens. Acoustic sensors and vibration sensors may measure acoustic noise and vibration, respectively, of machinery and other components within the diagnostic laboratory systems.

Collision sensors may generate data indicating the occurrence of collisions within the diagnostic laboratory systems. For example, collision sensors may generate data indicating collision of robotic arms and other moving components within diagnostic laboratory systems. Distance sensors and proximity sensors may determine relative locations of moving components, including specimen containers, within the diagnostic laboratory systems. Tactile sensors, which may be implemented as capacitive sensors, may generate data (e.g., signals) when a moving component within diagnostic laboratory systems.

As described above, diagnostic laboratory systems may contain a plurality of different sensors. The sensors improve the ability of the diagnostic laboratory systems to be automated and may improve the accuracy of specimen testing.

However, when one sensor malfunctions, the instrument or module containing the malfunctioning sensor may also malfunction. In some embodiments, the entire diagnostic laboratory system may become inoperable by the malfunction of a single module or component caused by the malfunction of a single sensor. As such, the diagnostic laboratory system or a module or instrument thereof may then be disabled or may at least operate at a reduced efficiency. Thus, improved diagnostic laboratory systems and methods of operating diagnostic laboratory systems with malfunctioning sensors are sought.

The diagnostic laboratory systems, modules, components, and methods described herein provide alternative senor configurations and/or usage upon malfunction or degradation of one or more sensors. A sensor check program determines the health or status of a first sensor, for example. If the status of the first sensor is degraded or if the first sensor has malfunctioned, a sensor-scaling program may reduce or eliminate reliance on the first sensor. The diagnostic laboratory system may utilize one or more second sensors to supplement or replace data of the first sensor. For example, sensor data generated by other sensors may be used during the process to supplement the data that would otherwise be generated by the first sensor. In other embodiments the data that would otherwise be generated by the first sensor may be imputed with data generated by one or more other sensors. Thus, the diagnostic laboratory systems and modules and instruments thereof may continue to operate when a sensor degrades or malfunctions.

In some embodiments, the diagnostic laboratory system may include a user-controllable, user configuration program that enables users to manually configure the sensor check program and/or the sensor-scaling program. The user configuration program may enable users of a diagnostic laboratory system to enable and/or disable specific sensors based on various operational factors such as performance accuracy, energy consumption, operation time, cost, and budget. In some embodiments, disabling a sensor means disregarding data generated by the sensor. In some embodiments, scaling sensor data includes at least partially disregarding sensor data generated by a sensor in response to the user input. Thus, if one or more sensors have degraded, a user may disable the one or more sensors instead of the replacing one or more sensors right away. For example, the degraded one or more sensors may be replaced at a time that will not cause the diagnostic laboratory system to cease performing analyses or at a time when a budget enables the one or more sensors to be replaced.

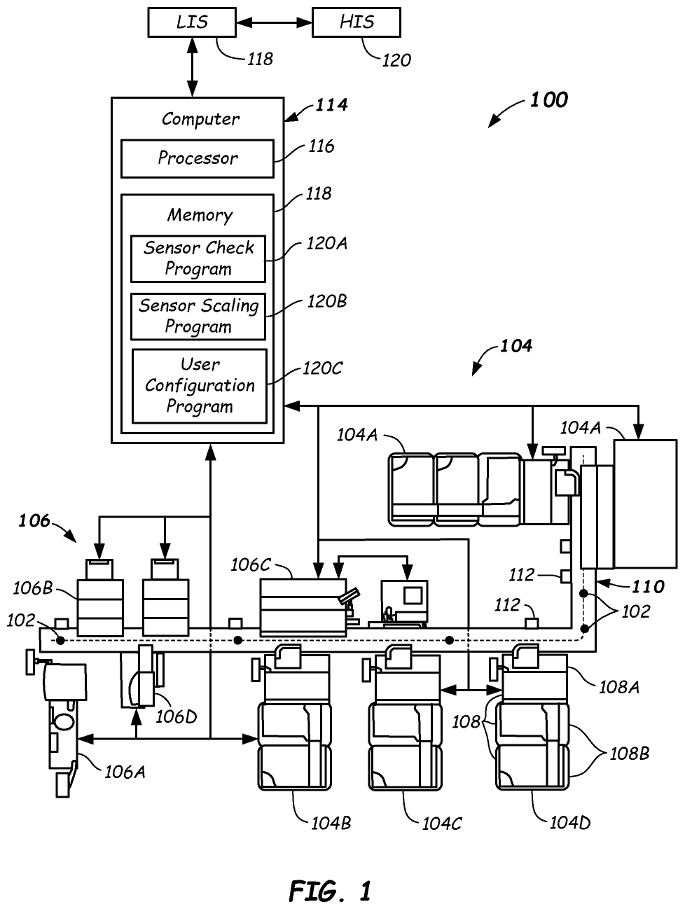

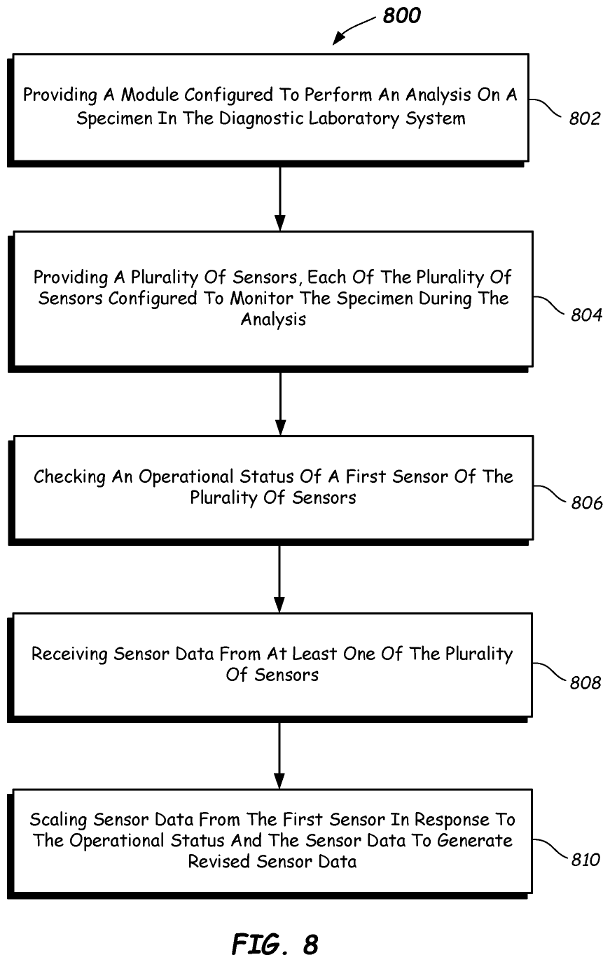

These and other diagnostic laboratory systems, components, modules, instruments, methods, and programs are described herein with reference to FIGS. 1-8.

Reference is now made to FIG. 1, which illustrates a block diagram of a diagnostic laboratory system 100 that performs tests and/or assays on biological specimens (e.g., biofluids). The tests and assays may be referred to as analyses performed on the specimens. The specimens may include blood serum, urine, and other liquids described above that are obtained from patients. The specimens are collected from the patients and stored in specimen containers 102 (a few labelled), which are configured to be transported throughout the diagnostic laboratory system 100. Test orders, which include tests and assays that are to be performed on the specimens, may be received electronically in the diagnostic laboratory system 100 as described herein.

The diagnostic laboratory system 100 may include a plurality of instruments 104 and modules 106 that may process the specimen containers 102 and perform testing on specimens located therein. In the embodiment of FIG. 1, the diagnostic laboratory system 100 includes four instruments 104, which are referred to individually as a first instrument 104A, a second instrument 104B, a third instrument 104C, and a fourth instrument 104D. The diagnostic laboratory system 100 may include a plurality of modules 106, some of which are labelled as a first module 106A, a second module 106B, a third module 106C, and a fourth module 106D. The instruments 104 may each include two or more modules (e.g., submodules), wherein some of the submodules may perform functions identical or similar to functions performed by one or more of the modules 106.

Reference is made to the fourth instrument 104D, which may be similar or identical to the other instruments. The fourth instrument 104D includes three submodules 108, which may include a processing module 108A and one or more analyzer modules 108B. The processing module 108A may prepare specimens for testing and may identify specimen containers received in the fourth instrument 108D. The analyzer modules 108B may perform testing on the specimens. The instruments 104 and modules 106 may include a plurality of sensors (not shown in FIG. 1) as described herein.

The diagnostic laboratory system 100 may include a track 110 configured to transport the specimen containers 102 or enable transport of the specimen containers 102 to and from instruments 104 and/or modules 106 in the diagnostic laboratory system 100. The track 110 may include, for example, a railed track (e.g., a monorail or a multiple rail), a collection of conveyor belts, conveyor chains, moveable platforms, magnetic transportation, or any other suitable type of conveyance mechanism. In some embodiments, the specimen containers 102 may be coupled to self-propelled devices, such as linear motors, that travel on the track 110.

In some embodiments, the diagnostic laboratory system 100 may include one or more position sensors 112 (a few labeled) located proximate the track 110. The position sensors 112 may read identifying indicia, such as barcodes, RFID labels, or the like, affixed to the specimen containers 102. Thus, the position sensors 112 may be able to provide information identifying the specimen containers 102 (and specimens therein) and the locations of the specimen containers 102 in the diagnostic laboratory system 100. For example, the locations of the specimen containers 102 may be determined by the position sensors 112 that have identified specific ones of the specimen containers 102.

The diagnostic laboratory system 100 may include a computer 114 that may be in communication with the instruments 104, the modules 106, the position sensors 112, and other sensors and components described herein. In some embodiments, the computer 114 may be proximate the instruments 104 and the modules 106 and in other embodiments, the computer 114 may be remote from the instruments 104 and modules 106. The computer 114 may include a processor 116 and memory 118, wherein the processor 116 executes programs comprising executable code that may be stored in the memory 118.

One of the programs stored in the memory 118 may be a sensor check program 120A that is configured to check the status of one or more of the sensors within the diagnostic laboratory system 100 as described herein. Another program may be a sensor-scaling program 120B that may be configured to scale sensor data generated by one or more of the sensors in response to data generated by the sensor check program 120A. Another program may be a user configuration program 120C that may be configured to enable users of the diagnostic laboratory system 100 to configure scaling of the sensors. For example, the sensor data may be scaled at least in part based on user input. The above-described programs are described in greater detail herein. The memory 118 may store one or more other programs.

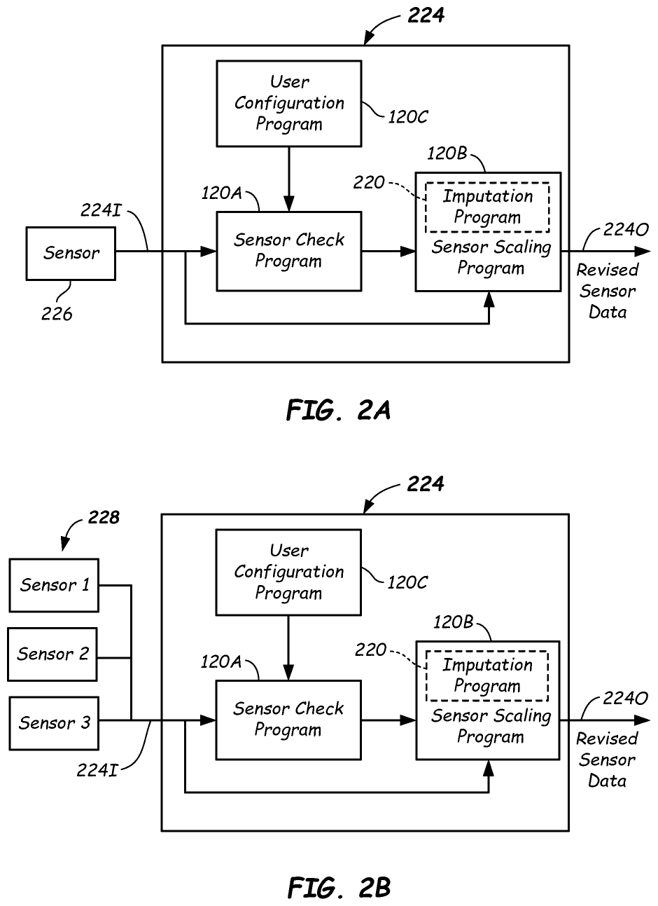

Additional reference is made to FIG. 2A, which illustrates a block diagram of a system 224, such as a sensor monitoring and revision system, showing an embodiment of the interaction of the sensor check program 120A, the sensor scaling program 120B, and the user configuration program 120C. The system 224 receives data from a sensor 226, which may be a single sensor, and manipulates the sensor data as described herein. The sensor 226 may be any of the sensors in the diagnostic laboratory system 100 described herein. In some embodiments, the sensor check program 120A, the sensor-scaling program 120B, and the user configuration program 120C may be implemented in a single program. For example, the above-described programs may be modules implemented in a single program that is stored in the memory 118 and executed by the processor 116.

The system 224 includes an input 2241 that receives sensor data generated by the sensor 226 within the diagnostic laboratory system 100 (FIG. 1). The sensor 226 illustrated in FIG. 2A may be a generic sensor and is representative of any of the sensors in the diagnostic laboratory system 100. In some embodiments, the sensor 226 may be an imaging device, a pressure sensor, a temperature sensor, a position sensor, a vibration sensor, voltage or current sensor, and the like, for example. The sensor 226 outputs sensor data indicative of a measured parameter, such as images, pressure, temperature, position, vibration, voltage, current, and/or other parameters that may be generated by the sensor 226.

The input 2241 may be coupled to the sensor check program 120A and the sensor-scaling program 120B. The input to the sensor check program 120A may be sensor data generated during a self-test of the sensor 226, for example. In other embodiments, the input to the sensor check program 120A may be sensor data generated during operation of the diagnostic laboratory system 100. In some embodiments, the sensor check program 120A may store the status of different sensors. When a particular sensor outputs sensor data to the system 224, the sensor check program 120A may output the operational status (operational status data) of the specific sensor to the scaling program 120B as described herein. The sensor-scaling program 120B may manipulate the sensor data in response to the operational status data generated by the sensor check program 120A as described herein.

In some embodiments, the operational status data generated by the sensor check program 120A may be a binary value indicating whether the sensor 226 is functioning properly or not. For example, when the sensor 226 outputs sensor data to the system 224, the sensor check program 120A may output sensor status data having a value of one (1) indicating that the sensor 226 is functioning properly or a value of zero (0) indicating that the sensor 226 is not operating properly. In some embodiments, the sensor check program 120A may receive data that is more descriptive of the status of the sensor 226. For example, the sensor check program 120A may receive data providing a percentage of the operability of the sensor 226 and/or data indicating one or more specific problems with the sensor 226.

In some embodiments, the sensor check program 120A may perform one or more tests on the sensor 226 or may cause the sensor 226 to perform one or more self-tests or other sensor diagnostics. In other embodiments, the sensor check program 120A may analyze sensor data generated by the sensor 226 to determine the status of the sensor 226. These and other embodiments are described in greater detail below.

The user configuration program 120C may enable users to set specific parameters of one or more sensors, such as the sensor 226. The sensor data or operational status data that would otherwise be input to or generated by the sensor check program 120A may be input (e.g., manually input) by a user of the diagnostic laboratory system 100 by way of the user configuration program 120C. For example, when a fault (malfunction) is detected with the sensor 226, the user may input user data regarding faults detected in the sensor 226 and/or other sensors via the user configuration program 120C. The user data may cause the system 224 to ignore sensor data generated by the sensor 226, scale the sensor data per the user input, or provide sensor data in lieu of sensor data that may be generated by the sensor 226. Embodiments of the user configuration program 120C are described in greater detail below.

The sensor-scaling program 120B may receive sensor data generated by the sensor 226 and sensor status generated by the sensor check program 120A. In response to the sensor data and the sensor status, the sensor-scaling program 120B may generate revised sensor data, which is output via the output 2240. The sensor-scaling program 120B may perform a plurality of operations to generate the scaled sensor data, such as manipulating or disregarding the sensor data as described in greater detail below.

In some embodiments, the system 224 may include an imputation program 220. In the embodiment of FIGS. 2A and 2B, the imputation program 220 is shown implemented in the sensor-scaling program 120B. However, the imputation program 220 may be a separate program or implemented elsewhere. The imputation program 220 may impute sensor data from nonfunctioning sensors. For example, should the sensor 226 malfunction, the imputation program 220 may impute the sensor data that would otherwise be generated by the sensor 226 based at least on part on sensor data received from other sensors, for example.

Additional reference is made to FIG. 2B, which illustrates an embodiment of the system 224 coupled to a plurality of sensors 228. In the embodiment of FIG. 2B, the system 224 may be coupled to three sensors, which are labelled individually as sensor 1, sensor 2, and sensor 3. Components within the system 224, such as the sensor check program 120A and the sensor-scaling program 120B, may determine which sensor is transmitting sensor data to the system 224 and may perform the above-described functions specific to the sensor or sensor type transmitting the sensor data. Thus, system 224 may interact with a plurality of sensors, which may be different types of sensors.

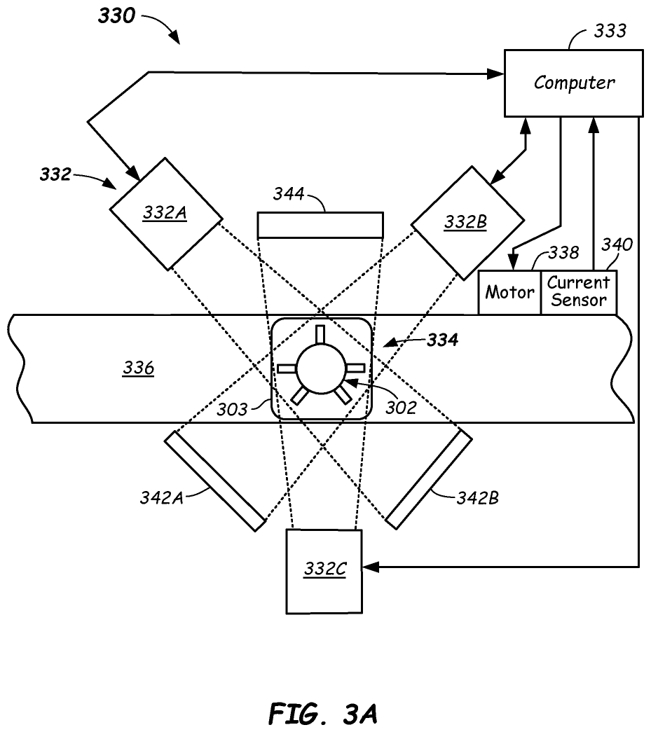

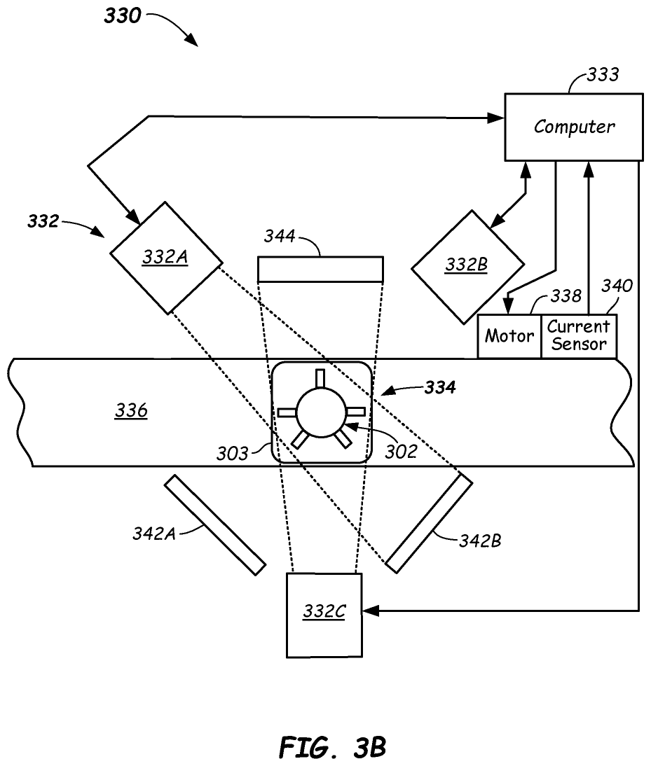

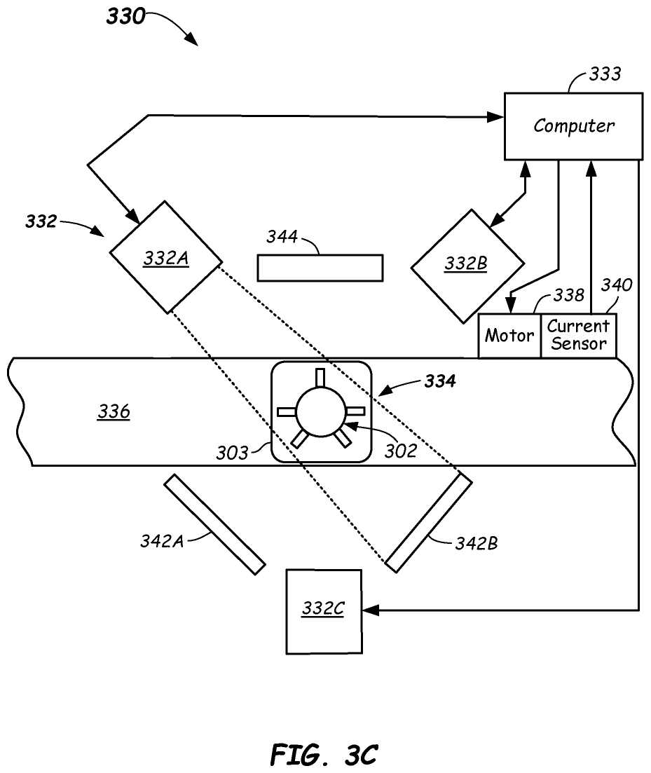

Additional reference is made to FIG. 3A, which illustrates a module 330, which may include a plurality of sensors. In the embodiment of FIG. 3A, the module 330 is implemented as an imaging module, such as an imaging module for checking for HIL or the presence of an artifact. The sensors located in the module 330 may operate in the same or a similar manner as the sensor 226 (FIG. 2A) and/or the sensors 228 (FIG. 2B). The module 330 may include a plurality of imaging devices 332 implemented as the sensor 226 and/or the sensors 228.

In the embodiment of FIG. 3A, the module 330 includes three imaging devices 332, referred to individually as a first imaging device 332A, a second imaging device 332B, and a third imaging device 332C. The imaging devices 332 may be configured to capture images of specimen containers (e.g., specimen container 302) that are transported through the module 330, such as on carriers (e.g., carrier 303). Other devices and methods may be used to transport the specimen container 302 to or through the module 330. The captured images are in the form of image data and may be processed as the sensor data described herein.

The imaging devices 332 generate image data representative of the specimen container 302 and/or the specimen located therein when the specimen container 302 is located in an imaging location 334. The imaging location 334 is a location within the module 330 wherein one or more of the imaging devices 332 may capture images of the specimen container 302 and/or a specimen located therein. As shown in FIG. 3A, the imaging devices 332 may be arranged around the imaging location 334 so as to capture images from a plurality of views of the specimen container 302. In the embodiment of FIG. 3A, the configuration of the imaging devices 332 enables a 360° view of the specimen container 302 to be captured. This imaging enables labels located on the specimen container 302 to be captured by at least one of the imaging devices 332. This imaging may further enable unobstructed images of the specimens to be captured, such as unobstructed views of the serum or plasma, for example.

Components in the module 330 may be controlled by a computer 333 that may also process sensor data (e.g., pixelated image data) generated by the module 330. The computer 333 may be local to the module 330 or remote from the module 330. In some embodiments, the computer 333 may be implemented in the computer 114 (FIG. 1). In some embodiments, the computer 333 may be in electronic communication with the computer 114 (FIG. 1). The computer 333 may process the image data generated by the imaging devices 332 and/or sensor data generated by other sensors in the module 330. In some embodiments, the computer 333 may facilitate the transmission of the image data generated by the imaging devices 332 and/or other sensors to the computer 114 (FIG. 1).

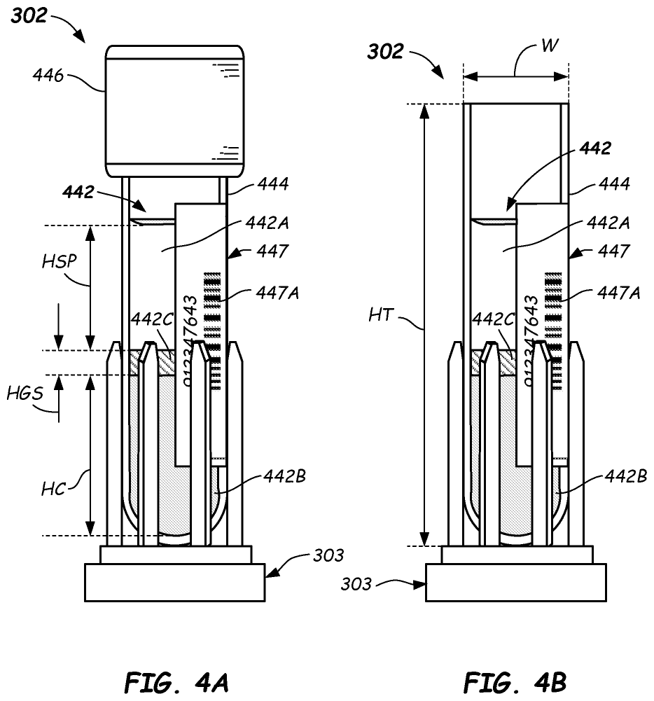

Additional reference is made to FIG. 4A, which illustrates a side elevation view of an embodiment of the specimen container 302 with a specimen 442 located therein. The specimen container 302 may include a tube 444 capped by a cap 446 that is configured to seal the tube 444. The specimen 442 shown in FIG. 4A has undergone a centrifuge process to separate components of the specimen 442. Heavier components in the specimen 442 have settled toward the bottom of the specimen container 302 and lighter components have risen toward the top of the tube 444 in response to the centrifuge process. In the embodiment of FIG. 4A, the specimen 442 may be a blood sample. During the centrifuge process, serum or plasma 442A may be separated from red blood cells 442B. A separator 442C (e.g., a gel separator) may separate the serum or plasma 442A from the red blood cells 442B.

The serum or plasma 442A is illustrated as having a height HSP, the separator 442C is illustrated as having a height HGS, and the red blood cells 442B is illustrated as having a height HC. In some embodiments, the serum or plasma 442A is analyzed (e.g., imaged), so the height HSP of the serum or plasma 442A may be measured. The height HSP may enable a processor or the like to determine the volume of the serum or plasma 442A in the specimen container 302. The height HSP also may be used to provide information to other modules as to the depth that a probe (pipette) may need to extend into the specimen container 302 to enable aspiration of the serum or plasma, for example.

The shape and/or color of the cap 446 may provide indications of the type of the specimen container 302 and/or chemicals located within the specimen container 302. One or more imaging devices (e.g., imaging devices 332—FIG. 3A) may generate color pixelated image data representative of the cap 446, which may be processed as sensor data as described herein. Software, for example, may analyze the image data to determine the color and/or shape of the cap 446. Other features of the cap 446 may be determined.

The specimen container 302 may have a label 447 affixed thereto. The label 447 may contain information indicative of the specimen 442, tests to be conducted on the specimen 442, and/or the type of specimen container 302. Information on the label 447 may be read by one or more imaging devices (e.g., imaging devices 332—FIG. 3A) and may generate image data representative of the label 447. Software may analyze the image data to read the information on the label 447. In the embodiment of FIG. 4A, the label 442 has a barcode 447A located thereon, which may be imaged by one or more imaging devices and read by the above-described software. Additional reference is made to FIG. 4B, which illustrates the specimen container 302 with the cap 446 removed. As shown in FIG. 4B, the tube 444 has a height HT and a width W, which both may be measured by analyzing image data generated by one or more of the imaging devices 332.

In addition to the other measurements made on the specimen container 302, the location of the specimen container 302 within the module 330 may also be measured by analyzing one or more images of the specimen container 302 within the module 330. In some embodiments, the pose of the specimen container 302 may also be measured by analyzing one or more images of the specimen container 302. In some embodiments, the position of the specimen container 302 within the carrier 303 may be measured by analyzing one or more images of the specimen container 302 and the carrier 303.

In some embodiments accurately calculating dimensions of the specimen container 302, the specimen 442, the tube 444, and other items imaged by the imaging devices 332, necessitates that the positions of the imaging devices 332 be known. For example, the imaging devices 332 may be set in specific or predetermined positions in the module 330 during a calibration process. If one or more of the imaging devices 332 move, the computer 333 and/or other computers may not be able to accurately calculate the above-described dimensions.

The module 330 may include a track 336 that moves the specimen container 302 and/or the carrier 303 or enables the specimen container 302 and/or the carrier 303 to be moved throughout the module 330. In some embodiments, the track 336 may include a conveyor, such as a belt (not shown), that moves the specimen container 302 and/or the carrier 303. In some embodiments, a motor 338 may be configured to move the conveyor or otherwise move the carrier 303 on the track 336. The motor speed and/or direction may be controlled by instructions generated by the computer 333. A current sensor 340 may measure current drawn by the motor 338 wherein the measured current may be received and/or processed by the computer 333. In some embodiments, the measured current may be processed as sensor data as described herein. Erratic current or excessive current drawn by the motor 338 may be an indication of a problem or imminent problem in the module 330. Optionally, a voltage sensor may be used.

The module 330 may include one or more light reflectors and/or illumination sources that provide light to the imaging devices 332. In the embodiment of FIGS. 3A-3C, the module 330 includes a first illumination source 342A, a second illumination source 342B, and a light reflector 344. The first illumination source 342A and the second illumination source 342B may emit light having predetermined intensities and wavelengths in response to instructions generated by the computer 333. The first illumination source 342A, the second illumination source 342B, and the light reflector 344 may be in predetermined and/or fixed locations within the module 330. For example, the positions of the first illumination source 342A, the second illumination source 342B, and the light reflector 344 may be set during assembly and/or calibration of the module 330. Optionally, light reflector 344 may also be an illumination source.

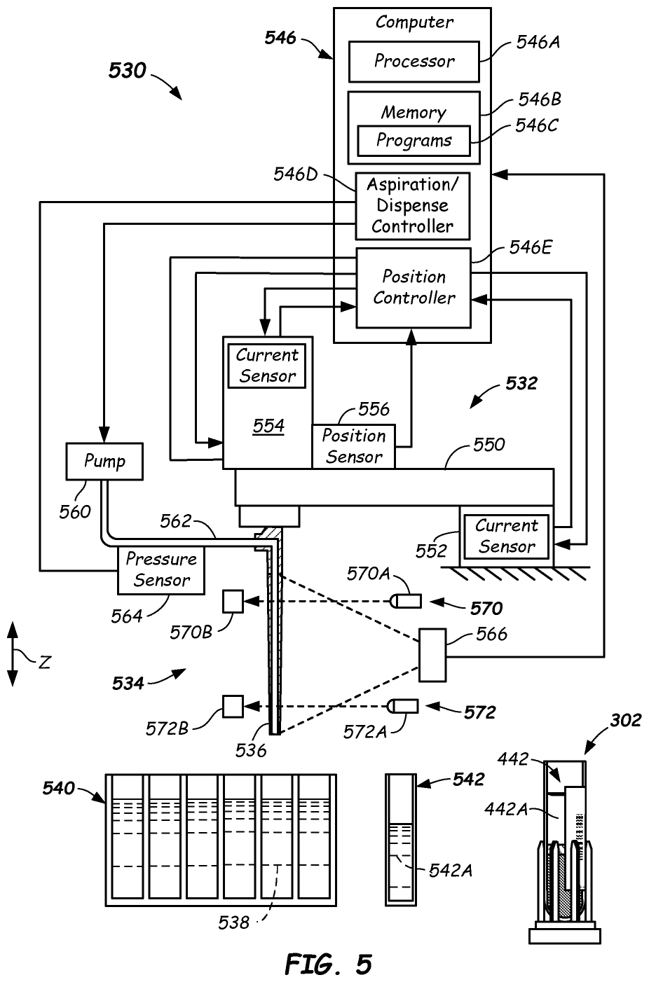

Additional reference is made to FIG. 5, which illustrates a block diagram of an embodiment of an aspiration and dispensing module 530, which may be referred to as an aspiration module herein. The aspiration and dispensing module 530 may be implemented in at least one of the instruments 104 (FIG. 1) and/or modules 120 (FIG. 1) of the diagnostic laboratory system 100 (FIG. 1). Other embodiments of the aspiration and dispensing module 530 may be used in the diagnostic laboratory system 100 (FIG. 1).

The aspiration and dispensing module 530 may aspirate and dispense specimens (e.g., specimen 442), reagents, and the like to enable the instruments 104 (FIG. 1) and modules (FIG. 1) to perform chemical analyses on the specimens 442 or simply aspirate and then re-dispense the specimen into a different container or vessel. The aspiration and dispensing module 530 may include a robot 532 that is configured to move a pipette assembly 534 within the aspiration and dispensing module 530. In the embodiment of FIG. 5, a probe (pipette) 536 of the pipette assembly 534 is shown preparing to aspirate a reagent 538 from a reagent packet 540. The specimen container 302 is shown in FIG. 5 with the cap 446 (FIG. 4) removed, which may have been performed by a decapping module (not shown). The pipette assembly 534 may be configured to position the probe 536 so as to aspirate the serum or plasma 442A from the specimen container 302.

The reagent 538, other reagents, and a portion of the serum or plasma 442A may be dispensed into a reaction vessel 542, such as a cuvette or other suitable container. The reaction vessel 542 is shown as being rectangular in cross-section. However, the reaction vessel 542 may have other shapes depending on analyses that are to be performed. In some embodiments, the reaction vessel 542 may be configured to hold a few microliters of liquid 542A. The cuvette 542 may be made of a material that passes light for photometric analysis by one or more imaging devices as described herein. In some embodiments, the material may pass light having a spectrum (e.g., wavelengths) from 180 nm to 2,000 nm, for example. It is noted that only a portion of the serum or plasma 442A may be dispensed into the reaction vessel 542 and other portions of the serum or plasma 442A may be dispensed into other reaction vessels (not shown). In addition, other reagents may be dispensed into the reaction vessel 542 together with possibly other liquids and/or magnetic particles.

Some components of the aspiration and dispensing module 530 may be electrically coupled to a computer 546. In the embodiment of FIG. 5, the computer 546 may include a processor 546A and memory 546B. Programs 546C may be stored in the memory 546B and executed on the processor 546A. The computer 546 may also include a position controller 546E and an aspiration/dispense controller 546D that may be controlled by programs, such as the programs 546C stored in the memory 546B. In some embodiments, the computer 546 and the components therein may be implemented in the computer 114 (FIG. 1). The position controller 546E and/or the aspiration/dispense controller 546D may be implemented in separate devices (e.g., computers) in some embodiments.

The programs 546C may include algorithms that control and/or monitor components within the aspiration and dispensing module 530, such as the position controller 546E and/or the aspiration/dispense controller 546D. As described herein, one or more of the components may include one or more sensors that may be monitored by one of the programs 546C. The programs 546C also may perform self-test routines on the sensors. The results of the self-test routines may be transmitted to the system 224 (FIGS. 2A-2B). Sensor data generated by the one or more sensors also may be transmitted to the system 224 (FIGS. 2A-2B).

The robot 532 may include one or more arms and motors that are configured to move the pipette assembly 534 within the aspiration and dispensing module 530. In the embodiment of FIG. 5, the robot 532 may include an arm 550 coupled between a first motor 552 and the pipette assembly 534. The first motor 552 may be electrically coupled to the computer 546 and may receive instructions generated by the position controller 546E. The instructions may instruct the first motor 552 as to direction and speed of the first motor 552. The first motor 552 may be configured to move the arm 550 to enable the probe 536 to aspirate and/or dispense specimens and/or reagents as described herein. The first motor 552 may include or be associated with a current sensor that is configured to measure current drawn by the first motor 552. Sensor data (e.g., measured current) generated by the current sensor may be transmitted to the computer 546.

A second motor 554 may be coupled between the arm 550 and the pipette assembly 534 and may be configured to move the probe 536 in a vertical direction (e.g., a Z-direction) to aspirate and/or dispense liquids as described herein. The second motor 554 may move the probe 536 in response to instructions generated by the programs 546C. For example, the second motor 554 may enable the probe 536 to enter into and retract from the specimen container 302, the reaction vessel 542, and/or the reagent packet 540. Liquids may then be aspirated and/or dispensed as described herein. The second motor 554 may include or be associated a current sensor that is configured to measure current drawn by the second motor 554. Sensor data (e.g., measured current) generated by the current sensor may be transmitted to the computer 546.

The aspiration and dispensing module 530 may include a position sensor 556. In the embodiment of FIG. 5, the position sensor 556 is mechanically coupled to the robot 532. In some embodiments, the position sensor 556 may be coupled to other components in the aspiration and dispensing module 530. The position sensor 556 may be configured to sense positions of one or more components of the robot 532 or other components within the aspiration and dispensing module 530, such as the pipette assembly 534. In the embodiment of FIG. 5, the position sensor 556 may measure the position of the arm 550, the pipette assembly 534, and/or the probe 536 and may generate position data that may be processed as the sensor data described herein. The position data may be transmitted to the computer 546 and may ultimately be sensor data input to the system 224 (FIGS. 2A-2B).

The aspiration and dispensing module 530 may also include a pump 560 mechanically coupled to a conduit 562 and electrically coupled to the aspiration/dispense controller 546D. The pump 560 may generate a vacuum or negative pressure (e.g., aspiration pressure) in the conduit 562 to aspirate liquids. The pump 560 may generate a positive pressure (e.g., dispense pressure) in the conduit 562 to dispense liquids. In some embodiments the pump 560 may comprise both a high-speed pump and a low speed pump.

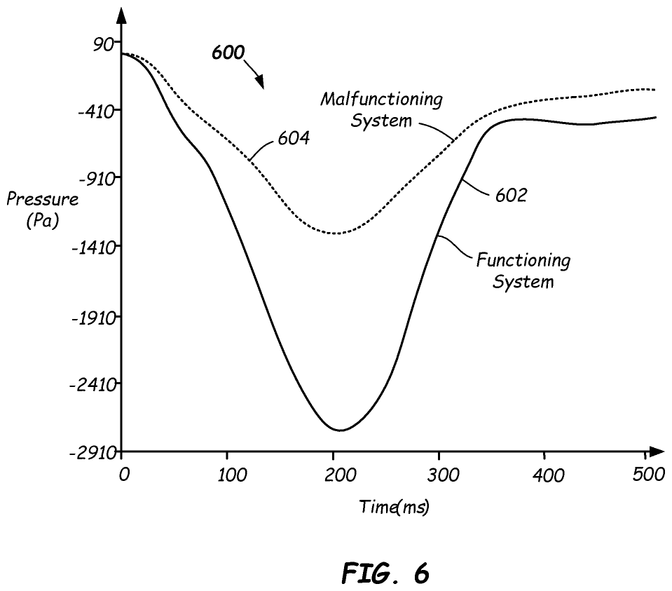

A pressure sensor 564 may be configured to measure pressure in the conduit 562 and generate pressure data indicative of the pressure. In some embodiments, the pressure sensor 564 may be configured to measure aspiration pressure and generate pressure data. In some embodiments, the pressure sensor 564 may be configured to measure dispense pressure and generate pressure data. The pressure data may be in the form of a pressure trace as a function of time and as described with reference to FIG. 6 below. The pressure data ultimately may be transmitted to the computer 114 (FIG. 1) and may be input to the system 224 as sensor data. For example, the sensor data may be used by the sensor check program 120A to determine the status of the pressure sensor 564.

Additional reference is made to FIG. 6, which is a graph 600 illustrating pressure traces of the pipette assembly 534 measured by the pressure sensor 564. In the embodiment of FIG. 6, the pressure traces show the pipette assembly 534 aspirating liquid and show a functioning system and a malfunctioning system. The malfunctioning system may be due to the pressure sensor 564 being faulty and/or the pump 560 being faulty. For illustration purposes, the malfunction will be described as being due to a faulty pressure sensor 564. The pressure trace 602 illustrates a trace of a functioning pressure sensor 564 that shows a high vacuum measured during aspiration. The pressure trace 604 illustrates a trace of a faulty pressure sensor 564 that does not measure the high vacuum during aspiration. For example, the pressure trace 604 shows a low vacuum, which may be indicative of a faulty pressure sensor 564. During a dispense operation, the faulty pressure sensor 564 may generate faulty pressure data. The pressure traces may be sensor data input to the system 224 (FIGS. 2A-2B).

Referring again to FIG. 5, the aspiration and dispense module 530 may include an imaging device 566 configured to capture images of the probe 536. For example, the probe 536 may be transparent so the imaging device 566 may capture images of liquids located in the probe 536. The captured images may comprise image data that is transmitted to and analyzed by the computer 546. The programs 546C may analyze the image data to determine the quality of the liquid in the probe 536. For example, the programs 546C may determine whether the liquid in the probe 536 has a bubble, bubbles, or a clot. The image data and/or a status of the imaging device 566 may be input to the system 224 (FIGS. 2A-2B) and processed as sensor data as described herein.

In some embodiments, the aspiration and dispense module 530 may include one or more imaging devices implemented as one or more optical sensors that may be configured to sense liquids in the probe 536. In the embodiment of FIG. 5, the aspiration and dispense module 530 may include a first optical sensor 570 and a second optical sensor 572. In some embodiments, the aspiration and dispense module 530 may include at least one of the first optical sensor 570, the second optical sensor 572, or the imaging device 566.

The first optical sensor 570 may include a first transmitter 570A and a first receiver 570B. The first transmitter 570A may include a light source, such as a laser or light-emitting diode (LED) that is configured to transmit light through the probe 536. The light passing through the probe 536 is received by the first receiver 570B. The first receiver 570B may be coupled to the computer 546 and may process image data generated by the first receiver 570B. The image data generated by the first receiver 570B may be data indicative of the intensity of light received by the first receiver 570B. The second optical sensor 572 may be identical or substantially similar to the first optical sensor 572 and may be located vertically spaced from the first optical sensor 572.

The image data generated by the first receiver 570B and/or the second receiver 572B may be generated as the probe 536 moves relative to the first receiver 570B and/or the second receiver 572B. Transitions in the image data may be indicative of transitions between air and liquid in the probe 536 and are indicative of the liquid level in the probe. For example, the probe 536 may transmit more light in areas where no liquids are present. By correlating the vertical position of the probe 536 with the transitions in the image data, the height of liquid in the probe 536 may be calculated.

During aspiration processes the pressure in the conduit 562 may be measured by the pressure sensor 564 to determine volume of the aspirated liquid. The imaging device 566 may be used to determine whether air bubbles are present in the aspirated liquid. The optical sensors 570, 572 may measure the height of the liquid in the probe 536. The methods of pressure sensing and imaging are complementary because the pressure sensing typically is not able to detect air bubbles inside the aspirated liquid and the imaging using the imaging device 566 is not able to obtain precise volume measurements due to meniscus at the surface of the aspirated liquid. When the pressure sensor 564, the optical sensors 570, 572, and the imaging device 566 are generating reliable data, the computer 546 and the diagnostic laboratory system 100 (FIG. 1) are confident about the volume of the aspirated liquid.

When fewer than all of the pressure sensor 564, the optical sensors 570, 572, or the imaging device 566 are available, the computer 546 and/or the diagnostic laboratory system 100 is able to detect severe deviation cases in the aspiration volume using the system 224 (FIGS. 2A-2B), which may be implemented in the computer 546. For example, the system 224 may rely on an assumption that majority of the pipettor operations will perform as expected.

Referring to FIGS. 2A-2B, the sensor check program 120A determines the status of one or more sensors in the diagnostic laboratory system 100. In some embodiments, other programs may perform diagnostic tests, such as self-tests, on one or more sensors 226, 228 and may generate diagnostic data. The sensor check program 120A may receive the diagnostic data and process the diagnostic data or transmit the diagnostic data to the sensor-scaling program 120B as the operational status data described herein. The diagnostic tests may be performed during calibration processes, for example.

In embodiments wherein the sensors 226, 228 include imaging devices, such as the imaging devices 332 (FIGS. 3A-3C), the optical sensors 570, 572, and/or the imaging device 566 (FIG. 5), the calibration and/or diagnostic tests may include capturing an image of a known object under predetermined conditions, such as predetermined lighting conditions. An analysis of image data representative of the image may be performed by the sensor check program 120A or another program to generate the diagnostic data. The operational status of the imaging devices may be determined in response to an analysis of the diagnostic data. In some embodiments, an imaging device may capture images of its surroundings over a period of time. The images may be compared to one another to determine if the imaging device is degrading or has degraded. The images may also be compared to one another to determine if the imaging device has moved. This position data may be used by the sensor check program 120A to generate the operational status data that is transmitted to the sensor-scaling program 120B.

The sensor check program 120A may analyze the diagnostic data to generate a status of an imaging device. In some embodiments, the sensor check program 120A may determine that the imaging device is completely nonfunctional and may transmit such operational status data to the sensor-scaling program 120B. For example, if no image data is received from an imaging device or if the image data is all the same value, the sensor check program 120A may send operational status data to the sensor scaling program 120B indicating that the imaging device is faulty or nonfunctional.

In some embodiments, the sensor check program 120A may determine that the imaging device is able to capture images, but the quality of the captured images is not optimal. For example, if the image is degrading or has degraded over a period of time, the sensor check program 120A may transmit the status of the imaging device to the sensor-scaling program 120B. In some embodiments, the sensor check program 120A may determine that the imaging device is generating blurred images and may transmit this data to the sensor-scaling program 120B.

As described above, the sensor-scaling program 120B receives sensor status from the sensor check program 120A and the sensor data generated by the sensor(s). The sensor-scaling program 120B may scale the sensor data and/or impute the sensor data to generate the revised sensor data. In some embodiments, the sensor-scaling program 120B may transmit one or more instructions to components within the diagnostic laboratory system 100 to manipulate or revise the sensor data as described in greater detail herein.

Referring again to FIG. 3A, in some embodiments, the sensor scaling program 120B may disable one or more of the imaging devices 332. As described herein, in some embodiments, the sensor-scaling program 120B may instruct the diagnostic laboratory system 100 to function with one or more of the imaging devices 332 disabled. Reference is made to FIG. 3B, which illustrates the module 330 with the second imaging device 332B disabled. Reference is also made to FIG. 3C, which illustrates the module 330 with the second imaging device 330B and the third imaging device 330C disabled. Thus, the sensor-scaling program 120B enables the module 330 to operate or at least partially operate without all the imaging devices 332 being enabled or functional as described herein.

Referring to FIG. 3B, center offset of the specimen container 302 may be estimated using two of the imaging devices 332. For example, the sensor-scaling program 120B may receive image data from the first imaging device 332A and the third imaging device 332C and perform triangulation to estimate the center offset of the specimen container 302. The center offset and other data generated with the two imaging devices active may be output by the sensor-scaling program 120B as the revised sensor data. Referring to FIG. 3C, the offset perpendicular to the viewing direction of the third imaging device 332C can be estimated and output from the scaling sensor program 120B as the revised sensor data.

In some embodiments, the sensor-scaling program 120B may use the imputation program 220 to impute the sensor data and generate the revised sensor data based on the imputed sensor data. For example, in some embodiments, the sensor check program 120A may determine that an imaging device, such as the second imaging device 332B, is not functional. In some embodiments, the imputation program 220 may run an algorithm that generates image data that would otherwise be generated by one imaging device based on image data generate by another imaging device. For example, the imputation program 220 may include a trained image-to-image synthesizer using artificial intelligence, such as an autoencoder, that constructs image data that would otherwise be generated by the second imaging device 332B based on image data generated by the first imaging device 332A and/or the third imaging device 332C. In some embodiments, the constructed image data is not complete and may not generate a complete image. The status of the constructed image may be transmitted to the computer 114 (FIG. 1), which may perform processing and/or analysis based on the incomplete image. In some embodiments, the autoencoder may include or be implemented with a neural network and/or a conditional adversarial network. Other artificial intelligence algorithms may be used.

In some embodiments, the sensor check program 120A may determine that an imaging device is capturing images that are blurred. Blurred images may be the result of dirt and/or liquids on a lens of the imaging device, for example. For example, in some embodiments, the sensor check program 120A may determine that the image data representative of images does not include a predetermined number of sharp transitions. In response to the sensor check program 120A detecting blurred images, the sensor-scaling program 120B may run the imputation program 220 to manipulate image data generated by the imaging device to sharpen the images.

In some embodiments, super-resolution methods may be used to sharpen blurred images. The methods may be identical or similar to super-resolution methods used to increase the resolution of low-resolution images that have been artificially generated by bilinear down sampling or by blurring resulting from by down sampling. The sharpened images may be processed as the revised image data.

In some embodiments, the image sharpening methods may include a two-stage process that first trains a high-to-low generative adversarial network (GAN) to learn how to degrade and downsample high-resolution images. Once trained, the output of the high-to-low GAN is used to train a low-to-high GAN for image super-resolution. The low-to-high GAN may be used to increase the quality of the blurred images. Other methods and/or algorithms may be used to decrease blur in the images. The resulting images (e.g., the resulting image data) may be the revised sensor output of the system 224 and may replace image data (sensor data) generated by the imaging device that is generating blurred images.

In some embodiments, the imputation program 220 may instruct a module, such as the module 330 (FIGS. 3A-3C) and/or the diagnostic laboratory system 100 to perform certain operations to keep the module and/or the diagnostic laboratory system 100 operational irrespective of a nonfunctional imaging device. In some embodiments with some types of modules, in response to an imaging device being nonfunctional, the imputation program 220 may generate instructions that cause the specimen container 302 (FIGS. 4A-4B) to be rotated. For example, the instructions may cause a robot or the like to rotate the specimen container 302. In some embodiments, the instructions may cause the specimen container 302 to be transported to a module that rotates the specimen container 302 and then returns the specimen container 302 to the module. One or more of the functional imaging devices may then capture images of the portion of the specimen container 302 that could not be captured by the nonfunctional imaging device. The revised sensor data output by the system 224 (FIGS. 2A-2B) may then be the image data generated by the functional imaging device (s).

The operation of the system 224 (FIGS. 2A-2B) will now be described in conjunction with the aspiration and dispense module 530 (FIG. 5). With additional reference to FIG. 2B, the sensors 228 may be the sensors in the aspiration and dispense module 530. For example, sensor 1 may be the imaging device 566, sensor 2 may be the pressure sensor 564, and sensor 3 may be the position sensor 556. Other sensors may be used, such as the optical sensors 570, 572. The sensor check program 120A may test each of the sensors 228 and/or receive sensor data generated by the sensors 228. The testing may determine the functionality of each of the sensors 228. In some embodiments, the sensor-scaling program 120B disregards sensor data from a first sensors (e.g., the pressure sensor or imaging device) in response to the operational status of the first sensor being less than a predetermined functionality. In some embodiments, the sensor data may be at least partially disregarding. In some embodiments, the sensor data may be manipulated or supplemented as described herein.

As described above, the pressure sensor 564 may measure the pressure in the conduit 562 during aspiration and/or dispense operations. The imaging device 566 and optical sensors 570, 572 may capture images of the probe 536 and/or liquids located in the probe 536 and may generate image data representative of the probe 536 and/or the liquids.

Diagnostic data related to the status of the pressure sensor 564 may be generated during calibration. For example, a pressure trace, such as the pressure traces in FIG. 6 may be generated by aspirating and/or dispensing a liquid having a predetermined viscosity. The resulting pressure trace may be the diagnostic data and may be transmitted to the sensor check program 120A. The status of the pressure sensor generating the pressure trace may be determined in response to an analysis of the diagnostic data. In some embodiments, the pressure traces may be analyzed over time to determine if the measured pressure is declining, which may be interpreted by the sensor check program 120A as faulty pressure sensor 564 or a leak in the conduit 562.

Diagnostic data related to the operation of the imaging device 566 also may be generated as described above with reference to the imaging devices 332 (FIGS. 3A-3C). In some embodiments, the diagnostic data or additional diagnostic data may be generated by imaging the probe 536 when the probe is in a predetermined location. For example, the second motor 554 may move the probe 536 to a predetermined position, which may be verified by the position sensor 556. Image data generated by the imaging device 566 should show the probe 536 in the predetermined position. If the probe 536 is not in the predetermined position, the imaging device 566 may have moved or the position sensor 556 may be defective. This diagnostic data may be transmitted to the system 224 (FIGS. 2A-2B) and processed by the system 224 as described herein.

Diagnostic data related to the operation of the optical sensors 570, 572 may be generated by moving the probe 536 out of the light path between the transmitters 570A, 572A and the receivers 570B, 572B, for example. The resulting image data may will have a predetermined value or be within predetermined limitations. The data may be communicated to the sensor check program 120A and/or the sensor-scaling program 120B.

In some embodiments, the operational status of the imaging device 566 and/or the optical sensors 570, 572 may be tested as described above. In some embodiments, calibration of the imaging device 566 may be used to determine the operational status of the imaging device 566 and may include capturing an image of a background. In some embodiments, the imaging device 566 may capture an image of a target that is located in a fixed position within the aspiration and dispense module 530. The imaging device 566 may continually capture images and analyze the image data to determine whether there have been any changes in the image. The images may be communicated to the sensor check program 120A to generate the operational status of the imaging device 566. If no changes are present in the images, the imaging device 566 has remained in the fixed location within the aspiration and dispense module 530. The sensor check program 120A may indicate that the operational status of the imaging device 566 is functional. If there is change between images, the amount of change may be analyzed by the sensor check program 120A to determine the operational status of the imaging device 566.

In some embodiments, the imaging device 566 may capture images of the probe 536 and/or other portions of the pipette assembly 534 over a period of time with the probe 536 and/or the pipette assembly 534 in the same position when each image is captured. If the image of the probe 536 and/or the pipette assembly 534 has moved relative to the imaging device 566 over time, the imaging device 566 may have moved within the aspiration and dispense module 530. In other cases, the position sensor 556 may not be functioning properly. The different images may be analyzed to determine the operational status of at least the imaging device 566 or the position sensor 556 as described above.

The position sensor 556 may be tested and related diagnostic data may be generated by the sensor check program 120A. For example, the computer 546 may generate instructions to move the pipette assembly 534 to a predetermined position. The imaging device 566 and/or one or more of the optical sensors 570, 572 may generate image data of the probe 536 and/or the pipette assembly 534 to determine if the probe 536 and/or the pipette assembly 534 is in the predetermined position. It is noted that the aforementioned test of the position sensor 556 may not be accomplished if the imaging device 566 and the optical sensors 570, 572 are not functioning or have moved within the aspiration and dispense module 530. The movement may be communicated to the sensor check module 120A as the diagnostic data and may be used to determine the operational status of the imaging device 566, the optical sensor 570, 572, and/or the position sensor 556.

The pressure sensor 564 may be tested as described above to determine the operational status of the pressure sensor 564. For example, the pipette assembly 534 may aspirate and/or dispense a liquid having a known viscosity and may measure the pressure in the conduit 562. The pressure data generated by the pressure sensor 564 may be communicated to the sensor check program 120A to determine the operational status of the pressure sensor 564.

The sensor check program 120A may receive and analyze the diagnostic data of the sensors 228. In response to the analysis, the sensor check program 120A may generate the operational status data of the sensors and may transfer the operational status data to the sensor-scaling program 120B as described herein. The sensor-scaling program 120B may scale the sensor data as described herein. For example, if the imaging device 566 is defective (e.g., nonfunctional), the sensor scaling program 120B may disregard, or at least partially disregard, the image data generated by the imaging device 566. If the position sensor 556 and the pressure sensor 564 are functional, the system 224 may output revised sensor data indicating that the image data is to be disregarded. The sensor-scaling program 120B may use the pressure sensor data to determine whether bubbles are present in the aspirated liquid.

If the pressure sensor 564 is defective or nonfunctional, the sensor-scaling program 120B may disregard the pressure sensor data. In some embodiments, the sensor-scaling program 120B may use image data representative of the probe 536 to calculate the volume of liquid in the probe 536. This calculated volume may be output as the revised sensor data of the pressure sensor 564 and may be noted as not being accurate.

If the position sensor 556 is defective or nonfunctional, the sensor-scaling program 120B may use image data generated by the imaging device 566 and/or the optical sensors 570, 572 to determine the position of the probe 536. For example, the optical sensors 570, 572 may identify the location of the bottom of the probe 536 or other areas of the probe 536. The sensor-scaling program 120B may output this probe position as the revised sensor data and may indicate that the revised sensor data may not be accurate.

As described above, the system 224 may include a user configuration program 120C that may enable users to input data regarding sensor status. For example, users, such as operators or technicians, of the diagnostic laboratory system 100 may be able to manually change sensor status. Such status changes include disregarding (e.g., uninstalling sensors), installing sensors, and setting scaling of specific sensors. In some embodiments, users may manually change the sensor status based on operational factors, such as performance accuracy, energy consumption, operation time, cost, and budget. For example, a user may decide to disable sensor data of a dysfunctional sensor rather than replace the dysfunctional sensor. In some embodiments, a user may scale sensor data by at least partially disregarding the sensor data in response to user input.

By using the revised sensor data described herein, the module for which the revised sensor data was generated may continue to operate. Thus, a failure of a sensor may not cause catastrophic failure of a module or an entire diagnostic laboratory system. Rather, the module and/or the diagnostic laboratory system may continue to operate in a limited capacity until the failed sensor is repaired.

In some embodiments, the sensor-scaling program 120B may determine specific sensor data to use to supplement, replace, or impute sensor data of nonfunctioning sensors. In some embodiments, tables or the like may be used to determine which sensor data may be used to supplement, replace, or impute the sensor data of nonfunctioning sensors.

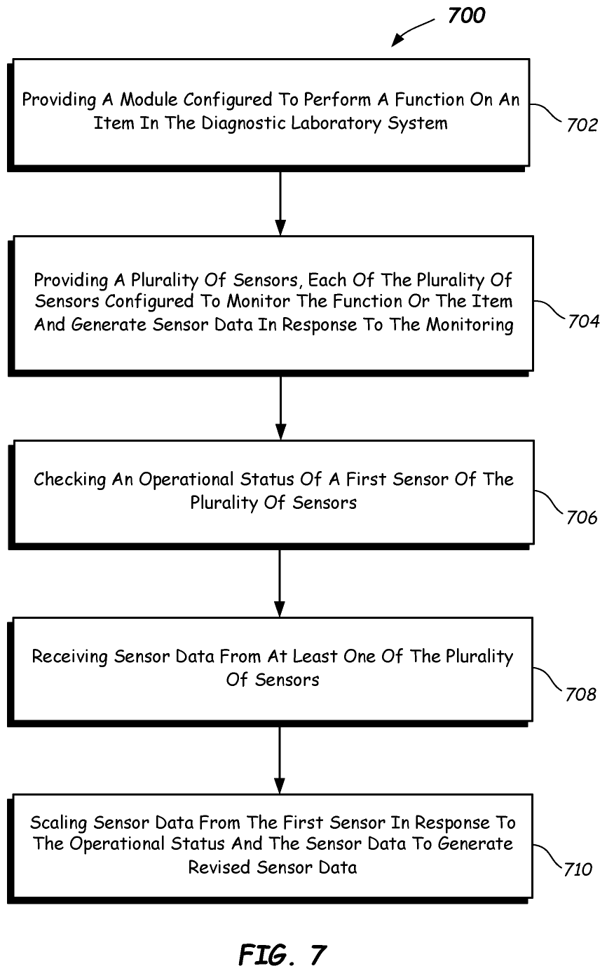

Reference is now made to FIG. 7, which is a flowchart illustrating a method 700 of operating a diagnostic laboratory system (e.g., diagnostic laboratory system 100). The method 700 includes, in 702, providing a module (e.g., module 330, module 530) configured to perform a function on an item (e.g., specimen 442, specimen container 302) in the diagnostic laboratory system. The method includes, in 704, providing a plurality of sensors (e.g., sensor 226, sensors 228), each of the plurality of sensors configured to monitor the function or the item and generate sensor data in response to the monitoring. The method 700 includes, in 706, checking an operational status of a first sensor of the plurality of sensors. The method 700 includes, in 708, receiving sensor data from at least one of the plurality of sensors. The method 700 includes, in 710, scaling sensor data from the first sensor in response to the operational status and the sensor data to generate revised sensor data.

Reference is now made to FIG. 7, which is a flowchart illustrating a method 800 of operating a diagnostic laboratory system (e.g., diagnostic laboratory system 100). The method 800 includes, in 802 providing a module (e.g., module 330, module 530) configured to perform an analysis on a specimen (e.g., specimen 442) in the diagnostic laboratory system. The method 800 includes, in 804, providing a plurality of sensors (e.g., sensor 226, sensors 228), each of the plurality of sensors configured to monitor the specimen during the analysis. The method 800 includes, in 806, checking an operational status of a first sensor of the plurality of sensors. The method 800 includes, in 808, receiving sensor data from at least one of the plurality of sensors. The method 800 includes, in 810, scaling sensor data from the first sensor in response to the operational status and the sensor data to generate revised sensor data.

While the disclosure is susceptible to various modifications and alternative forms, specific method and apparatus embodiments have been shown by way of example in the drawings and are described in detail herein. It should be understood, however, that the particular methods and apparatus disclosed herein are not intended to limit the disclosure but, to the contrary, to cover all modifications, equivalents, and alternatives falling within the scope of the claims.

What is claimed is:

1. A method of operating a diagnostic laboratory system, comprising:
providing a module configured to perform a function on an item in the diagnostic laboratory system;
providing a plurality of sensors, each of the plurality of sensors configured to monitor the function of the item and generate sensor data in response to monitoring;
checking an operational status of a first sensor of the plurality of sensors;
receiving sensor data from at least one of the plurality of sensors; and
scaling sensor data from the first sensor in response to the operational status and the sensor data to generate revised sensor data.

2. The method of claim 1, comprising performing at least a portion of the function using the revised sensor data.

3. The method of claim 1, comprising monitoring the item using the revised sensor data.

4. The method of claim 1, wherein the scaling comprises imputing sensor data from the first sensor in response to the operational status of the first sensor being less than a predetermined functionality.

5. The method of claim 1, wherein the scaling comprises imputing sensor data of the first sensor of the plurality of sensors in response to the operational status of the first sensor being nonfunctional.

6. The method of claim 5, wherein the imputing comprises imputing data of the first sensor using data from one or more second sensors.

7. The method of claim 1, wherein the scaling comprises disregarding sensor data from the first sensor in response to the operational status of the first sensor being less than a predetermined functionality.

8. The method of claim 1, wherein:
providing a module comprises comprising providing a module configured to analyze a specimen;
providing a plurality of sensors comprises providing at least one imaging device configured to capture images, the images comprising image data; and
generating sensor data comprises capturing one or more images using the at least one imaging device.

9. The method of claim 8, wherein checking an operational status of at least one of the plurality of sensors comprises analyzing image data generated by the at least one imaging device.

10. The method of claim 8, wherein scaling sensor data comprise disregarding image data generated by the at least one imaging device in response to checking an operational status of the at least one imaging device.

11. The method of claim 8, wherein:
checking an operational status of the at least one imaging device comprises determining whether image data generated by the at least one of the at least one imaging device is representative of a blurred image; and
scaling sensor data comprises manipulating the image data to sharpen the blurred image.

12. The method of claim 1, wherein providing a module comprises providing an aspiration module configured to aspirate a liquid.

13. The method of claim 12, wherein:
providing a plurality of sensors comprises providing a pressure sensor configured to generate sensor data indicative of aspiration pressure in response to the aspiration module aspirating a liquid; and
generating sensor data comprises generating pressure data using the pressure sensor in response to the aspiration module aspirating a liquid.

14. The method of claim 13, comprising generating a pressure trace using the pressure sensor and wherein checking an operational status of at least one of the plurality of sensors comprises analyzing the pressure trace.

15. The method of claim 13, wherein scaling sensor data comprises disregarding pressure data generated by the pressure sensor.

16. The method of claim 12, wherein:
providing a plurality of sensors further comprises providing at least one imaging device configured to capture images of an aspirated liquid, wherein the images comprise image data; and
generating sensor data comprises generating image data representative of images of the aspirated liquid.

17. The method of claim 16, wherein checking an operational status of at least one of the plurality of sensors comprises analyzing image data generated by at least one imaging device.

18. The method of claim 16, wherein providing at least one imaging device comprises providing at least one optical sensor.

19. The method of claim 16, wherein scaling sensor data comprises disregarding pressure data generated by a pressure sensor.

20. The method of claim 16, wherein scaling sensor data comprises disregarding image data generated by the imaging device.

21. The method of claim 16, wherein scaling sensor data comprises:
disregarding image data generated by a first imaging device;
generating first image data of the item using a second imaging device;
moving the item; and
generating second image data of the item using the second imaging device.

22. The method of claim 1, wherein scaling the sensor data comprises scaling sensor data based at least in part on a user input.

23. The method of claim 1, comprising receiving user input related to the operational status of the first sensor and wherein scaling sensor data comprises at least partially disregarding sensor data generated by the first sensor in response to the user input.

24. The method of claim 1, comprising receiving user input related to the operational status of the first sensor and wherein scaling sensor data comprises at least partially scaling sensor data generated by the first sensor based on the user input.

25. The method of claim 1, wherein the item is a specimen or specimen container.

26. A diagnostic laboratory system, comprising:
a module configured to perform a function on an item in the module;
a plurality of sensors, each of the plurality of sensors configured to monitor the function or the item and generate sensor data in response to monitoring; and
a computer configured to:
check an operational status of a first sensor;
receive sensor data from at least one of the plurality of sensors; and
scale sensor data generated by the first sensor in response to the operational status and the sensor data to generate revised sensor data.

27. The diagnostic laboratory system of claim 26, wherein the computer is further configured to impute sensor data of the first sensor in response to the operational status and the sensor data.

28. The diagnostic laboratory system of claim 26, wherein at least one of the plurality of sensors is a pressure sensor configured to measure pressure of a liquid during an aspiration process or a dispense process.

29. The diagnostic laboratory system of claim 26, wherein at least one of the plurality of sensors is an imaging device configured to capture an image of the item, the image comprising image data.

30. The diagnostic laboratory system of claim 26, wherein the computer is configured to receive user input, and wherein the computer is configured to scale the sensor data in response to the user input.

31. The diagnostic laboratory system of claim 26, wherein the computer is configured to:
receive user input related to the operational status of the first sensor; and
scale sensor data by at least partially disregarding sensor data generated by the first sensor in response to the user input.

32. The diagnostic laboratory system of claim 26, wherein the computer is configured to:
receive user input related to the operational status of the first sensor; and
scale sensor data generated by the first sensor at least partially based on the user input.

33. A method of operating a diagnostic laboratory system, comprising:
providing a module configured to perform an analysis on a specimen in the diagnostic laboratory system;
providing a plurality of sensors, each of the plurality of sensors configured to monitor the specimen during the analysis;
checking an operational status of a first sensor of the plurality of sensors;
receiving sensor data from at least one of the plurality of sensors; and
scaling sensor data from the first sensor in response to the operational status and the sensor data to generate revised sensor data.
